(19) United States
(12) Patent Application Publication
(10) Pub. No.: US 2016/0287280 A1
(43) Pub. Date: Oct. 6, 2016

(54) Peritoneal Peeling Surgical Instrument

(71) Applicant: JAPANESE ORGANIZATION FOR MEDICAL DEVICE DEVELOPMENT, INC., Tokyo (JP)

(72) Inventors: TAKAYUKI ASAO, Maebashi-shi (JP); HIROSHI HONDA, Funabashi-shi (JP)

(21) Appl. No.: 15/086,018

(22) Filed: Mar. 30, 2016

Related U.S. Application Data
(60) Provisional application No. 62/141,829, filed on Apr. 1, 2015.

(51) Int. Cl. A61B 17/32

(52) U.S. Cl. CPC A61B 17/32; A61B 2017/320044

(57) ABSTRACT

The present invention has been made with the aim to provide a surgical instrument, which can be put into a space between a peritoneum and muscle via a tunnel formed at the peritoneum easily and can be pulled strongly in a direction to peel the peritoneum from the muscle in hernia repair surgery or the like. A peritoneal peeling surgical instrument, which is put between muscle and a peritoneum via a tunnel formed at the peritoneum to peel the peritoneum from the muscle, includes: an operation part to be grasped and operated by the user; a shaft part connected with the operation part; a hinge part disposed at the tip of the shaft part; and a hook part connected with the shaft part via the hinge part. In response to operation to the operation part, the hook part is bent at the hinge part with respect to the shaft part and the bending state is fixed in one or more steps. The hook part has: a convex curved surface located at a bending direction side to contact the peritoneum; a flat surface located at a bending direction opposite side to contact the muscle; and a streamline tip part corresponding to the tip of the hook part.

### RELATED APPLICATION

This application claims the benefit of US provisional application entitled “Peritoneal Peeling Surgical Instrument” filed Apr. 1, 2015 having Ser. No. 62/141,829, the disclosure of which is herein incorporated in its entirety.

### TECHNICAL FIELD

The present invention relates to a peritoneal peeling surgical instrument. Specifically, a peritoneal peeling surgical instrument of the present invention is put into a space between muscle and a peritoneum via a tunnel formed at the peritoneum and is used for peeling the peritoneum from the muscle in hernia repair surgery or the like.

### BACKGROUND ART

Hernia is a disease in which tissue of a living body or an organ such as an internal organ protrudes from a portion where the tissue or organ is originally to exist through a laceration part of muscular tissue or a peritoneum that accommodates the tissue or organ. When the tissue or organ protruded by hernia compresses a nerve, this may cause a large pain or numbness. An example of common hernia is inguinal hernia or intervertebral disk hernia. Hernia can be repaired by surgery. For example, in hernia repair surgery, a treatment is performed to push the protruded tissue or organ back to a portion where the tissue or organ is originally to exist and then reinforce a damaged muscular tissue and a laceration part of a peritoneum. Moreover, in hernia repair surgery, another treatment may be performed to peel a peritoneum from muscular tissue endoscopically so as to remove hernia.

In hernia repair surgery, laparoscopic surgery is often performed. The laparoscopic surgery is surgery wherein a doctor inserts treatment instruments such as a forceps and a laparoscope through holes formed at a plurality of parts of body surface, and operates the treatment instruments from outside of the body so as to treat/remove an affected part while checking an image of the inside of the peritoneal cavity obtained by the laparoscope with a monitor. Laparoscopic surgery, which does not require large laparotomy and applies little load on the patient, realizes minimally invasive treatment. However, in a case where an affected part such as hernia exists at a deep part in the peritoneal cavity, an internal organ located between the body surface side and the affected part may disturb access of the laparoscope or the treatment instrument to the affected part in the laparoscopic surgery. Accordingly, in such laparoscopic surgery, it is required to move the disturbing internal organ transversely (exclusion) and secure an operational field in the body cavity. A baculiform instrument called a retractor is known as an instrument to achieve such exclusion of an internal organ (Patent Literature 1).

### CITATION LIST

### Patent Literatures

Patent Literature 1: JP 2002-360582 A

### SUMMARY OF INVENTION

### Technical Problem

As described above, hernia repair surgery includes a treatment to peel a peritoneum from muscular tissue so as to remove hernia. In this process, it is required to form a small opening (tunnel) at the peritoneum, put the tip of a retractor into a space between muscular tissue and a peritoneum via the tunnel, and peel the peritoneum from the muscular tissue while pulling the peritoneum strongly. However, a conventional retractor (Patent Literature 1) has a structure wherein an exclusion surface having a substantially rectangular shape is formed at the tip of a baculiform member. It is therefore difficult to put a conventional retractor into a tunnel formed at a peritoneum in the first place. Moreover, even when the tip of the retractor can be put into the tunnel, it is also difficult to pull the peritoneum with the retractor strongly enough to peel the peritoneum from the muscular tissue. A conventional retractor is therefore unsuitable for a treatment to peel a peritoneum from muscle.

Accordingly, the present invention has been made with the aim to provide a surgical instrument, which can be put into a space between a peritoneum and muscle via a tunnel formed at the peritoneum easily and can be pulled strongly in a direction to peel the peritoneum from the muscle in hernia repair surgery or the like.

### Solution to Problem

The present invention is basically constructed in a manner such that a baculiform hook part is connected with the tip of a baculiform shaft part and the hook part can be bent with respect to the shaft part after putting the hook part into a space between a peritoneum and muscle through a tunnel. This makes it possible to pull the peritoneum strongly from the inside thereof using the hook part in a bent state. Moreover, in the present invention, the hook part can be used to pull the peritoneum strongly and has a minimally invasive shape which applies no load on the body of a patient. Specifically, the present invention has the following structure.

The present invention relates to a peritoneal peeling surgical instrument 100.

The peritoneal peeling surgical instrument 100 of the present invention is put into a space between muscle and a peritoneum via a tunnel formed at the peritoneum and is used for peeling the peritoneum from the muscle.

The peritoneal peeling surgical instrument 100 of the present invention is provided with an operation part 10, a shaft part 20, a hinge part 30, and a hook part 40 for peeling.

The operation part 10 is gripped and operated by a user. The shaft part 20 is connected with the operation part 10. The hinge part 30 is disposed at the tip of the shaft part 20. The hook part 40 for peeling is connected with the shaft part 20 via the hinge part 30.

In such a structure, the peritoneal peeling surgical instrument 100 is constructed in a manner such that the hook part 40 is bent at the hinge part 30 with respect to the shaft part 20 in response to operation to the operation part 10 by the user and the bending state of the hook part 40 is fixed in one or a plurality of steps.

Moreover, the hook part 40 is shaped to have: a convex curved surface 41 located at a bending direction side which can be in contact with a peritoneum; a flat surface 42 located at a bending direction opposite side which can be in contact with muscle; and a streamline tip part 43 corresponding to the tip of the hook part 40.

As described above, the hook part 40 which can rotate on the hinge part 30 is disposed at the tip of the peritoneal peeling surgical instrument 100. This makes it possible to pull a peritoneum of a patient strongly with the hook part 40, by putting the hook part 40 into a space between the peritoneum and muscle via a tunnel and then bending the hook part 40 with respect to the shaft part 20. Consequently, hernia repair surgery can be achieved speedily and efficiently by a laparoscopic method. Moreover, regarding the hook part 40 of the peritoneal peeling surgical instrument 100, a portion to be in contact with a peritoneum is constituted of the convex curved surface 41, a portion to be in contact with muscle is constituted of the flat surface 42, and the tip part 43 corresponding to the tip has a streamline shape. Therefore, since the convex curved surface 41 of the hook part 40 comes into contact with the peritoneum, it is possible to prevent generation of damage or laceration even when the peritoneum is pulled strongly. Moreover, since the flat surface 42 is formed at the muscle side of the hook part 40, the hook part 40 rarely comes into contact with the muscle, and therefore it becomes possible to avoid damaging a muscle fiber. Furthermore, since the tip part 43 of the hook part 40 has a streamline shape, it is possible to put the hook part 40 into a space between the peritoneum and the muscle smoothly, and it is also possible to avoid damaging the peritoneum or muscle. Consequently, minimally invasive hernia repair surgery can be realized.

In the peritoneal peeling surgical instrument 100 of the present invention, it is preferable that the bending state of the hook part 40 can be fixed in at least a first step bent by 120±10° with respect to the shaft part 20, and a second step bent by 90±10° with respect to the shaft part 20.

As described above, since the hook part 40 can be fixed in a plurality of steps, it becomes easier to put the hook part 40 into a space between the peritoneum and the muscle from various directions so as to pull the peritoneum.

In the peritoneal peeling surgical instrument 100 of the present invention, it is preferable that the hinge part 30 has a slide shaft 31. The slide shaft 31 is a baculiform member having one end part connected with the operation part 10 and the other end part rotatably connected with the hook part 40. The slide shaft 31 can be advanced and retracted along the shaft part 20 in response to operation to the operation part 10 by the user. That is, it is possible to advance or retract the slide shaft 31 along the shaft part 20 by operating the operation part 10.

Here, in the peritoneal peeling surgical instrument 100, the connection state between the shaft part 20 and the hook part 40 is released by advancing the slide shaft 31. This puts the hook part 40 into a rotatable and freely movable state (a state where the hook part 40 moves freely centering on a rotation fulcrum). On the other hand, in the peritoneal peeling surgical instrument 100, the shaft part 20 and the hook part 40 are connected with each other by retracting the hinge part 30. This fixes the hook part 40 to the shaft part 20.

As described above, by separating the connection state between the shaft part 20 and the hook part 40 as the slide shaft 31 is advanced so that the hook part 40 becomes able to move freely, the bending angle of the hook part 40 can be set freely in accordance with the situation in the peritoneal cavity, the orientation of the surgery instrument or the like. Moreover, by putting the shaft part 20 and the hook part 40 into contact with each other as the slide shaft 31 is retracted so that the bending state of the hook part 40 is fixed (locked), the hook part 40 can be put into a suitable bending state. Consequently, the structure of the hinge part 30 described above improves the convenience of the peritoneal peeling surgical instrument 100.

The peritoneal peeling surgical instrument 100 of the present invention preferably has rigidity to withstand a load even in a case where a load of 500 g is applied to the tip part of the hook part 40 in the process of peeling a peritoneum in a state where the hook part 40 is fixed to the shaft part 20.

A load to be generated by pulling a peritoneum is approximately 500 g in hernia repair surgery. Therefore, a peritoneal peeling surgical instrument 100 which withstands a load of 500 g is sufficiently practical. Especially, a peritoneal peeling surgical instrument 100 preferably withstands a load of 500 g to 1,000 g in the process of peeling a peritoneum.

In a peritoneal peeling surgical instrument 100 of the present invention, a connection pin 32 configured to rotatably connect the slide shaft 31 and the hook part 40 with each other is preferably formed at a position deviated from a central shaft C of the slide shaft 31.

As described above, since the connection pin 32 configured to connect the slide shaft 31 and the hook part 40 with each other is deviated from the central shaft C of the slide shaft 31, the strength of the peritoneal peeling surgical instrument 100 can be further improved.

### Advantageous Effects of Invention

It is possible with the present invention to provide a surgery instrument, which can be put into a space between a peritoneum and muscle via a tunnel formed at the peritoneum easily and can be pulled strongly in a direction to peel the peritoneum from the muscle in hernia repair surgery or the like.

### BRIEF DESCRIPTION OF DRAWINGS

FIGS. 1a to 1c are plan views illustrating a peritoneal peeling surgical instrument according to the present invention;

FIGS. 2a to 2d are schematic views illustrating the shape of a hook part of a peritoneal peeling surgical instrument;

FIG. 3 is a schematic view illustrating the structure of a hinge part of a peritoneal peeling surgical instrument;

FIGS. 4a to 4e are schematic views illustrating the motion of a hook part of a peritoneal peeling surgical instrument;

FIGS. 5a to 5d are schematic views illustrating the motion of a hook part of a peritoneal peeling surgical instrument in an enlarged manner;

FIGS. 6a and 6b are schematic views illustrating an improvement example of a hinge part of a peritoneal peeling surgical instrument;

FIGS. 7a and 7b are schematic views illustrating another embodiment of a hinge part of a peritoneal peeling surgical instrument;

FIGS. 8a and 8b are schematic views illustrating another embodiment of a hinge part of a peritoneal peeling surgical instrument; and

FIG. 9 shows photographs illustrating a usage example of a peritoneal peeling surgical instrument.

### DESCRIPTION OF EMBODIMENTS

The following description will explain some embodiments for implementing the present invention with reference to the drawings. The present invention is not limited to the following embodiments but includes other embodiments to be provided by adding suitable modification that is obvious to those skilled in the art to the following embodiments.

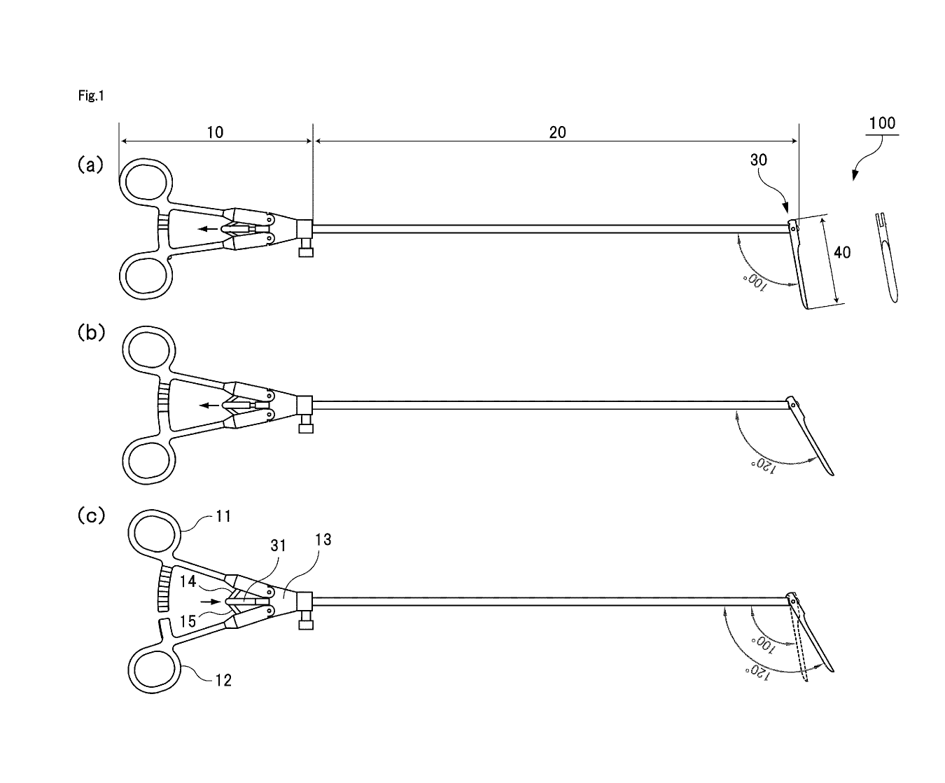

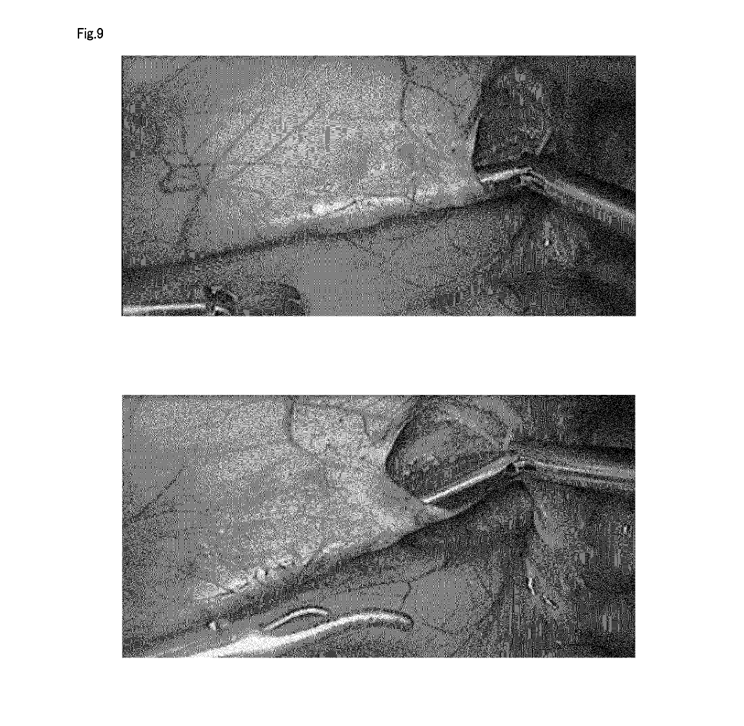

FIGS. 1a and 1c are plan views illustrating a peritoneal peeling surgical instrument 100 according to an embodiment of the present invention. As illustrated in FIGS. 1a and ac, the peritoneal peeling surgical instrument 100 comprises an operation part 10, a shaft part 20, a hinge part 30 and a hook part 40. Photographs illustrating a usage example of the peritoneal peeling surgical instrument 100 illustrated in FIGS. 1a and 1c are placed in FIG. 9 as a reference. As illustrated in FIG. 9, the peritoneal peeling surgical instrument 100 can be used suitably for surgery to put the hook part 40 disposed at the tip of the peritoneal peeling surgical instrument 100 into an opening (tunnel) formed at a peritoneum and then pull the peritoneum in a direction to be peeled from muscle. As illustrated in FIG. 9, regarding the peritoneal peeling surgical instrument 100, the bending state of the hook part 40 can be fixed at an arbitrary angle, and the fixing state of the hook part 40 withstands a load to be generated by peeling the peritoneum. Consequently, the peritoneal peeling surgical instrument 100 of the present invention can be used suitably in hernia repair surgery, for example. A detailed description will hereinafter be given of a specific structure of the peritoneal peeling surgical instrument 100.

The operation part 10 is a portion to be gripped by a user (e.g., surgeon). The user can rotate the hook part 40 on the hinge part 30 by operating the operation part 10. Thus, the operation part 10 composes a rotation mechanism of the hook part 40 together with the shaft part 20, the hinge part 30 and the hook part 40. In the embodiment illustrated in FIGS. 1a and 1c, the operation part 10 has a scissors-type structure. Specifically, the operation part 10 has: ring-shaped handle members 11 and 12 provided respectively with a round hole which a finger can enter; and a fulcrum member 13 which rotatably connects the tips of the pair of right and left handle members 11 and 12 with each other. With such a structure, by opening and closing the right and left handle members 11 and 12 with the fulcrum member 13 as a fulcrum, the opening and closing motion is transmitted through the shaft part 20 to the hinge part 30 and the hook part 40 disposed at the tip of the peritoneal peeling surgical instrument 100, so that the hook part 40 rotates with the hinge part 30 as a fulcrum.

More specifically, crank members 14 and 15 are disposed respectively at positions of the right and left handles members 11 and 12 close to the fulcrum member 13 in the operation part 10. The crank members 14 and 15 are rotatably connected with a slide shaft 31 which is inserted in the shaft part 20 and the fulcrum member 13. The tip of the slide shaft 31 is connected with the hook part 40 as will be described later. Thus, the slide shaft 31 has a tip connected with the hook part 40 and the other end connected with the crank members 14 and 15. When the right and left handle members 11 and 12 are opened, the slide shaft 31 is advanced to be extruded via the crank members 14 and 15 toward the tip side of the instrument 100 (see FIG. 1c). Thus, when the slide shaft 31 is advanced, the fixing state of the hook part 40 disposed at the tip of the peritoneal peeling surgical instrument 100 is released, and the hook part 40 rotates freely. On the other hand, when the right and left handle members 11 and 12 are closed, the slide shaft 31 is retracted to be pulled to the side closer to the user via the crank members 14 and 15 (see FIGS. 1a and 1b). Thus, when the slide shaft 31 is retracted, the hook part 40 disposed at the tip of the peritoneal peeling surgical instrument 100 is fixed to be in an unrotatable state. By adjusting the angle of the hook part 40 with respect to the shaft part 20 in this process, the hook part 40 can be fixed to be in a state bent with respect to the shaft part 20. For example, FIG. 1a illustrates a case where the hook part 40 is fixed in a state where the angle with respect to the shaft part 20 is 100°. Moreover, FIG. 1b illustrates a case where the hook part 40 is fixed in a state where the angle with respect to the shaft part 20 is 120°. Thus, by operating the operation part 10, the fixing angle of the hook part 40 can be set in two steps or more, or preferably in three steps or more.

Here, regarding the peritoneal peeling surgical instrument 100 of the present invention, the structure of the operation part 10 is not limited to the structure illustrated in FIGS. 1a to 1c. That is, the operation part 10 may have a structure other than the scissors-type structure. For example, the operation part 10 may have any structure as long as the slide shaft 31 can be advanced and retracted. For example, the operation part 10 may be of rotary grip type which is rotated by user's hand, though this example is not illustrated in the drawings. A rotary grip type is a structure wherein the slide shaft 31 can be retracted when being rotated in one direction and the slide shaft 31 can be advanced when being rotated in the other direction. An operation part 10 of such rotary grip type also can be employed as a preferable embodiment of the present invention.

The shaft part 20 is a baculiform portion to be used for ensuring a length to put the hook part 40, which is to be used for peeling a peritoneum, into a peritoneal cavity of a patient. Regarding the shaft part 20, an end part at a side closer to the user is fixed to the fulcrum member 13 of the operation part 10. It is to be noted that the shaft part 20 is fixed to the fulcrum member 13 in an unmovable state so as not to move or rotate even when the operation part 10 is operated (e.g., opened or closed). Moreover, in this embodiment, the shaft part 20 is constituted of a hollow cylindrical member. The slide shaft 31 is put into the hollow shaft part 20. The slide shaft 31 is advanced and retracted inside the shaft part 20 and along the shaft part 20. The length of the shaft part 20 can be adjusted suitably according to the use in surgery or the like. For example, the length of the shaft part 20 may be 10 cm to 30 cm, 15 cm to 25 cm or the like. Moreover, the peritoneal peeling surgical instrument 100 may be provided with an adjustment mechanism configured to freely adjust the length of the shaft part 20.

The hinge part 30 is a portion to be used for rotatably connecting the hook part 40 with the tip of the shaft part 20. The hinge part 30 is constructed to have a function to bend the hook part 40 with respect to the shaft part 20 and fix the bending state of the hook part 40 at least temporarily. Any structure having such a function may be employed as the structure of the hinge part 30. In this embodiment, the slide shaft 31 is employed as an example of the hinge part 30. An example of the structure of the hinge part 30 provided with the slide shaft 31 will be described later in detail.

The hook part 40 is a portion to be put into a space between muscle and a peritoneum via a tunnel formed at the peritoneum and pulled in a direction to peel the peritoneum from the muscle. The hook part 40 is disposed at the tip of the peritoneal peeling surgical instrument 100. Specifically, the hook part 40 is rotatably connected with the shaft part 20 via the hinge part 30. The hook part 40 is bent at the hinge part 30 with respect to the shaft part 20 in response to operation to the operation part 10. Moreover, the hook part 40 is constructed in a manner such that the bending state is fixed in one or a plurality of steps. In the example illustrated in FIG. 1A, the hook part 40 is fixed in a state where the bending angle with respect to the shaft part 20 is 100°. In the example illustrated in FIG. 1B, the hook part 40 is fixed in a state where the bending angle with respect to the shaft part 20 is 120°. Thus, the hook part 40 can preferably be fixed in at least a first step bent by 120±10° with respect to the shaft part 20 and a second step bent by 90±10° with respect to the shaft part 20. It is to be noted that the rotational range of the hook part 40 is not limited especially. For example, the rotational range of the hook part 40 may be set in a manner such that the angle with respect to the shaft part 20 falls within 90° to 180°.

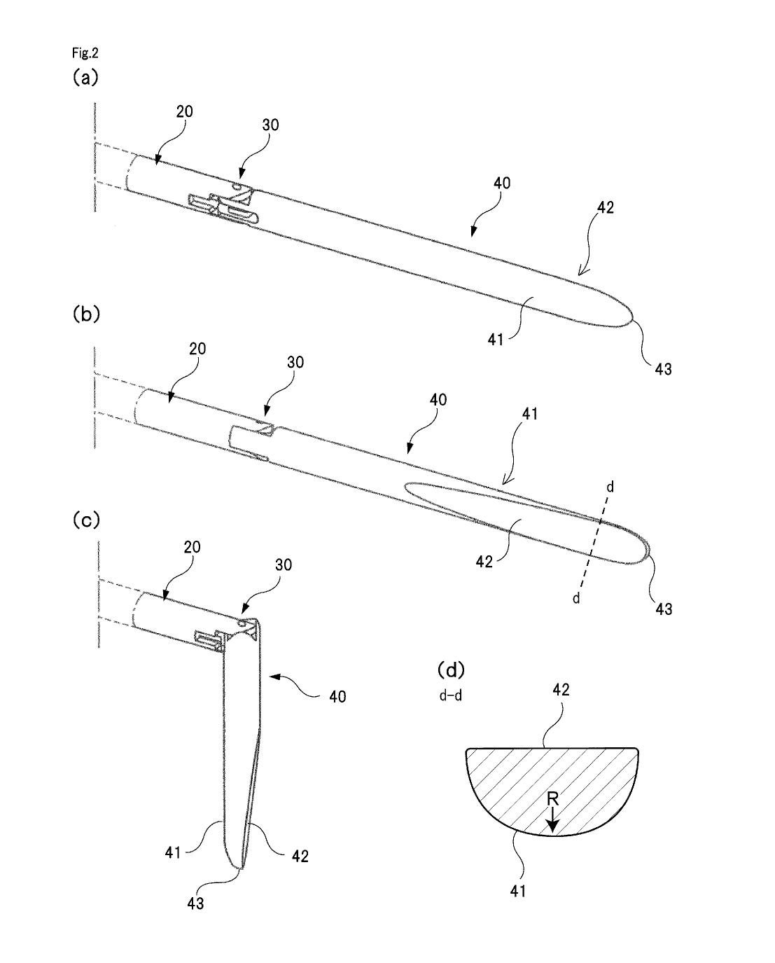

FIGS. 2a to 2d are enlarged views illustrating a specific shape of the hook part 40. FIG. 2a is an enlarged view wherein the hook part 40 is viewed from a surface which can be in contact with a peritoneum (surface at a bending direction side). FIG. 2b is an enlarged view wherein the hook part 40 is viewed from a surface which can be in contact with a muscle (surface at a bending direction opposite side). FIG. 2c is an enlarged view illustrating a state where the hook part 40 is bent. FIG. 2d illustrates a section cut along the line d-d illustrated in FIG. 2b.

As illustrated in FIGS. 2a to 2d, the hook part 40 is shaped to have: a convex curved surface 41 located at a bending direction side which can be in contact with a peritoneum; a flat surface 42 located at a bending direction opposite side which can be in contact with muscle; and a streamline tip part 43 corresponding to the tip of the hook part 40.

As illustrated in FIGS. 2a to 2d, a surface of the hook part 40 at a side, which comes into contact with a peritoneum when being put into a space between the peritoneum and the muscle, i.e., a surface at an bending direction inner side is constituted of the convex curved surface 41. It is preferable that the radius of curvature (R) of the convex curved surface 41 illustrated in FIG. 2d is approximately 2 mm to 10 mm, for example. Thus, since a surface at a side to be in contact with a peritoneum is constituted of the convex curved surface 41, it is possible to prevent generation of damage or laceration at the peritoneum even when the curved surface 41 is put into contact with the peritoneum and the peritoneum is pulled strongly. This makes it possible to put the hook part 40 into a space between a peritoneum of a patient and muscle via a tunnel and then pull the peritoneum of the patient strongly using the hook part 40 in a bent state.

The flat surface 42 is formed in a surface of the hook part 40 at a side to be in contact with muscle when being put into a space between a peritoneum and the muscle, i.e., a surface at a bending direction opposite side. Whole area of the surface at a bending direction opposite side is not required to be the flat surface 42 but at least a part of the surface is required to be the flat surface 42. Here, whole area of the surface at a bending direction opposite side may be the flat surface 42. In the example illustrated in FIGS. 2a to 2d, the flat surface 42 is formed partially in a region from the tip to the middle of the hook part 40. The length of the flat surface 42 may be, for example, 40% to 100%, 45% to 90% or 50% to 80% of the whole length of the hook part 40. Moreover, the length of the flat surface 42 may be at least 10 mm, and can be 10 mm to 60 mm or 20 mm to 40 mm. Since the muscle side of the hook part 40 is constituted of the flat surface 42, the hook part 40 rarely comes into contact with muscle and it becomes possible to avoid a situation that a muscle fiber is damaged.

The tip part 43 of the hook part 40 has a streamline shape. “Streamline tip part 43” means that the tip part 43 configured to join the curved surface 41 at a bending direction inner side and the flat surface 42 at a bending direction opposite side with each other has a rounded shape. “Streamline tip part 43” does not include a case where the tip part of the hook part 40 is cut perpendicularly to have a level surface or a case where the tip part of the hook part 40 is sharpened into a needle shape. Thus, since the tip part 43 of the hook part 40 has a streamline shape, it becomes possible to put the hook part 40 into a space between a peritoneum and muscle smoothly without damaging the peritoneum and the muscle.

As described above, the hook part 40 includes: the convex curved surface 41 located at a bending direction side which can be in contact with a peritoneum; the flat surface 42 located at a bending direction opposite side which can be in contact with muscle; and the streamline tip part 43 corresponding to the tip of the hook part 40. This makes it possible to realize minimally invasive surgery using the peritoneal peeling surgical instrument 100.

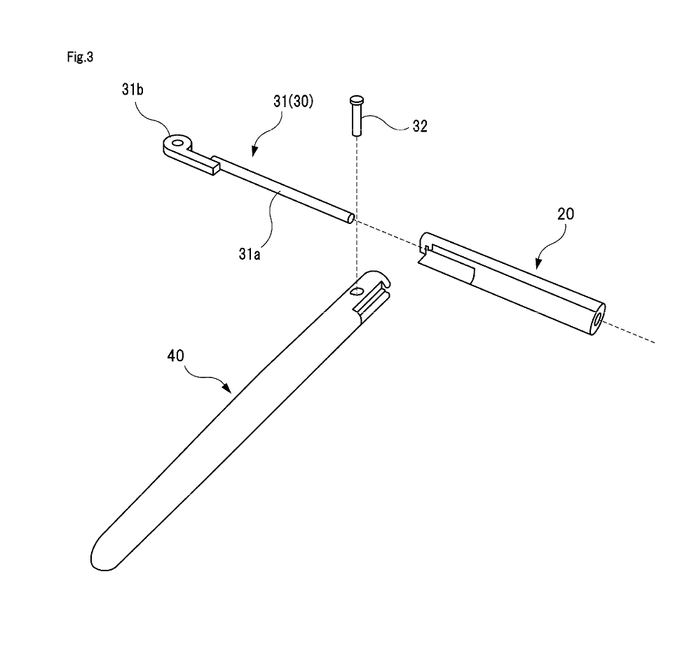

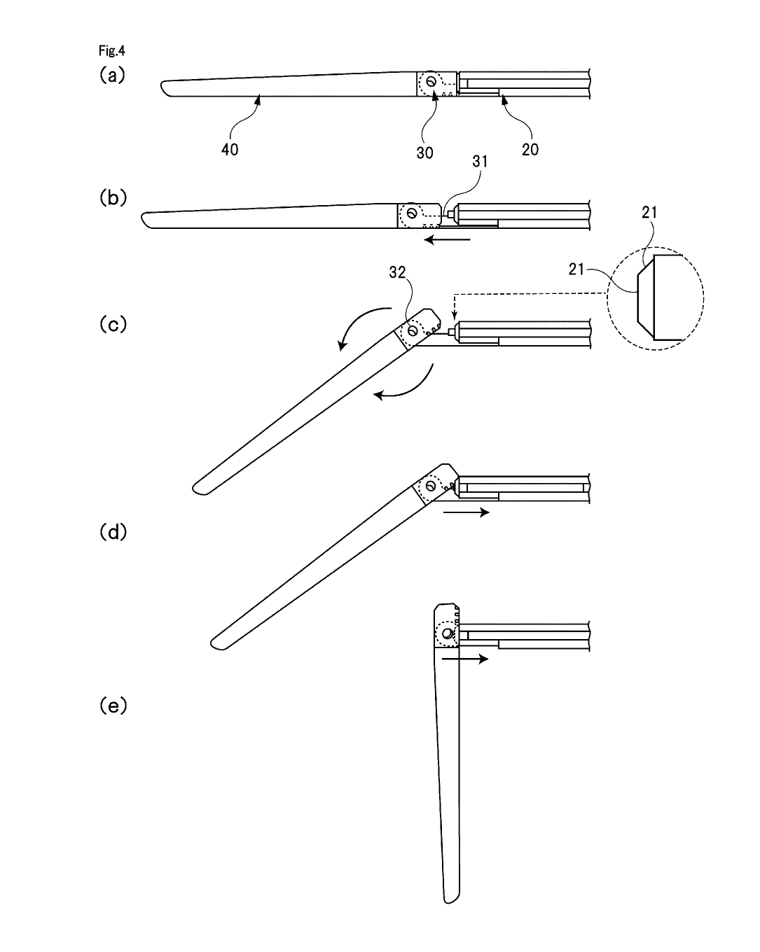

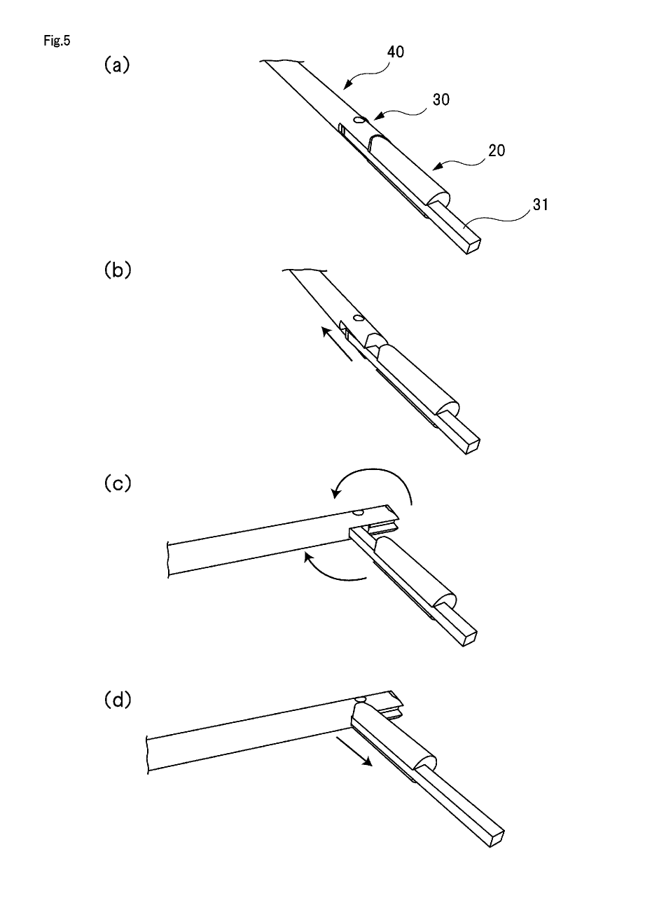

Next, a preferable embodiment of the hinge part 30 will be explained with reference to FIGS. 3 to 5d. FIG. 3 is a schematic view extracting and illustrating elements which compose the hinge part 30 and the peripheral portion thereof. FIGS. 4a to 4e illustrate the motion of the hook part 40 via the hinge part 30. FIGS. 5a to 5d are enlarged views wherein the hook part 40 is viewed from another angle.

As illustrated in FIG. 3, the hinge part 30 has the slide shaft 31 and a connection pin 32. It is to be noted that the slide shaft 31 is essentially a long shaft member extending along the longitudinal direction (dotted line) thereof, though the slide shaft 31 in FIG. 3 is cut schematically and drawn short. As illustrated in FIG. 3, the slide shaft 31 is put into the hollow cylindrical shaft part 20 and is advanced and retracted forward and backward along the shaft part 20. Moreover, the slide shaft 31 has an elongate cylindrical shaft part 31a, and a ring part 31b having a round hole disposed at the tip of the cylindrical shaft part 31a. It is to be noted that the other end of the cylindrical shaft part 31a of the slide shaft 31 is connected with the operation part 10 of the peritoneal peeling surgical instrument 100 as is clear from FIGS. 1a to 1c, though this is not illustrated in FIG. 3. This allows the slide shaft 31 to advance and retract forward and backward in the center hole of the shaft part 20 in conjunction with operation to the operation part 10.

The ring part 31b of the slide shaft 31 is connected with the base end side of the hook part 40 by the connection pin 32. That is, a round hole is formed at a side surface of the base end side of the hook part 40. Thus, the connection pin 32 is put in so as to communicate the round hole of the ring part 31b with the round hole of the hook part 40 in a state where the ring part 31b of the slide shaft 31 is put into the base end side of the hook part 40. This rotatably connects the hook part 40 with the tip (ring part 31b) of the slide shaft 31, with the connection pin 32 as a rotation fulcrum.

After the slide shaft 31 is inserted into the shaft part 20 and the hook part 40 is attached to the tip of the slide shaft 31, the state illustrated in FIG. 4a is obtained. In the state illustrated in FIG. 4a, the hook part 40 is not bent and is parallel to the shaft part 20. In the state illustrated in FIG. 4a, the slide shaft 31 is pulled to a side closer to the user by operation to the operation part 10. This firmly puts the hook part 40, which is attached to the tip of the slide shaft 31, into contact with the tip of the shaft part 20. This fixes the connection state between the hook part 40 and the shaft part 20.

Next, after the operation part 10 is operated so that the slide shaft 31 is advanced toward the tip side thereof, the hook part 40 is separated from the tip of the shaft part 20 as illustrated in FIG. 4b. That is, the shaft part 20 does not move, while the hook part 40 disposed at the tip of the slide shaft 31 moves in a direction to be separated from the shaft part 20. This forms a space between the hook part 40 and the shaft part 20, so that the fixing state between the hook part 40 and the shaft part 20 is released. Such a motion of the slide shaft 31 interlocks with a motion of the operation part 10 at a side closer to the user. For example, when the pair of right and left handle members 11 and 12 which compose the operation part 10 are opened, the slide shaft 31 is advanced to be extruded toward the tip side as illustrated in FIG. 1c. This operates the hook part 40 so as to be separated from the shaft part 20.

When the fixing state between the hook part 40 and the shaft part 20 is released as illustrated in FIG. 4c, the hook part 40 becomes able to freely rotate with the connection pin 32 at the tip of the slide shaft 31 as a rotation fulcrum. In a state where fixation between the hook part 40 and the shaft part 20 is released, the movable range of the hook part 40 is preferably set so that the angle with respect to the shaft part 20 falls within 90° to 180°, for example.

Next, when the operation part 10 is operated so that the slide shaft 31 is retracted toward a side closer to the user as illustrated in FIG. 4d, the hook part 40 is again firmly put into contact with the tip of the shaft part 20. This puts the hook part 40 into a state fixed to the tip of the shaft part 20. When the slide shaft 31 is retracted in a state where the hook part 40 is bent at a predetermined angle with respect to the shaft part 20, the hook part 40 is fixed to the tip of the shaft part 20 while maintaining the bending state. Consequently, the bending state of the hook part 40 is maintained as illustrated in FIG. 4d. In the state illustrated in FIG. 4d, the hook part 40 is bent by approximately 135±10° with respect to the shaft part 20, for example.

Similarly to FIG. 4d, FIG. 4e illustrates a state where the hook part 40 is fixed to the tip of the shaft part 20. In the example illustrated in FIG. 4e, the hook part 40 is bent by approximately 90±10° with respect to the shaft part 20. As illustrated in FIGS. 4d and 4E, the bending angle of the hook part 40 of the peritoneal peeling surgical instrument 100 of the present invention to be fixed to the shaft part 20 is preferably set in multiple steps.

An operation method of the operation part 10 for fixing the hook part 40 to the tip of the shaft part 20 as illustrated in FIGS. 4d and 4e can be understood by referring to FIGS. 1a and 1b. That is, when the pair of right and left handle members 11 and 12 which compose the operation part 10 are closed, the slide shaft 31 is pulled to the side closer to the user via the crank members 14 and 15. This puts the hook part 40, which is connected with the tip of the slide shaft 31, into contact with the shaft part 20 and fixes the hook part 40.

Moreover, it is also possible to devise the tip of the shaft part 20 in order to stabilize the fixing state between the hook part 40 and the shaft part 20. For example, as illustrated in FIG. 4c, a level surface 21 and an inclined surface 22 are formed at the tip part of the shaft part 20. The level surface 21 is located at the tip of the shaft part 20, and is a flat surface perpendicular to the axial direction of the shaft part 20. The inclined surface 22 is formed around the level surface 21, and is an inclined surface which becomes gradually wider with a distance from the level surface 21. Thus, the level surface 21 and the inclined surface 22 are disposed at the tip of the shaft part 20. For example, in the state of FIG. 4d, a side surface of the hook part 40 comes into contact with the inclined surface 22 at the tip of the shaft part 20. Thus, since the side surface of the hook part 40 comes into contact with the inclined surface 22 of the shaft part 20, the fixing state between the hook part 40 and the shaft part 20 can be stabilized. Moreover, in the state of FIG. 4d, a side surface of the hook part 40 comes into contact with the level surface 21 at the tip of the shaft part 20. Thus, since a side surface of the hook part 40 comes into contact with the level surface 21 of the shaft part 20, the fixing state between the hook part 40 and the shaft part 20 can be stabilized. This can stabilize the bending state of the hook part 40 with respect to the shaft part 20 in multiple steps.

FIGS. 5a to 5d illustrate the motion to bend the hook part 40 in an enlarged manner. FIG. 5a illustrates a state where the hook part 40 is fixed to the tip of the shaft part 20 without being bent. FIG. 5b illustrates a state where the slide shaft 31 is advanced toward the tip side so that the hook part 40 is separated from the shaft part 20. FIG. 5c illustrates a case where the fixing state between the hook part 40 and the shaft part 20 is released and the hook part 40 rotates freely. Furthermore, FIG. 5d illustrates a state where the slide shaft 31 is retracted toward a side closer to the user after the hook part 40 is bent, so that the hook part 40 and the shaft part 20 are connected again with each other and fixed. As illustrated in FIG. 5d, the bending state of the hook part 40 with respect to the shaft part 20 is stable. Even when a load of approximately 500 g is applied to the tip of the hook part 40 in the state illustrated in FIG. 5d, for example, the bending state of the hook part 40 is maintained. Therefore, it becomes possible to put the hook part 40 into a space between a peritoneum and muscle and then pull the peritoneum strongly using the hook part 40.

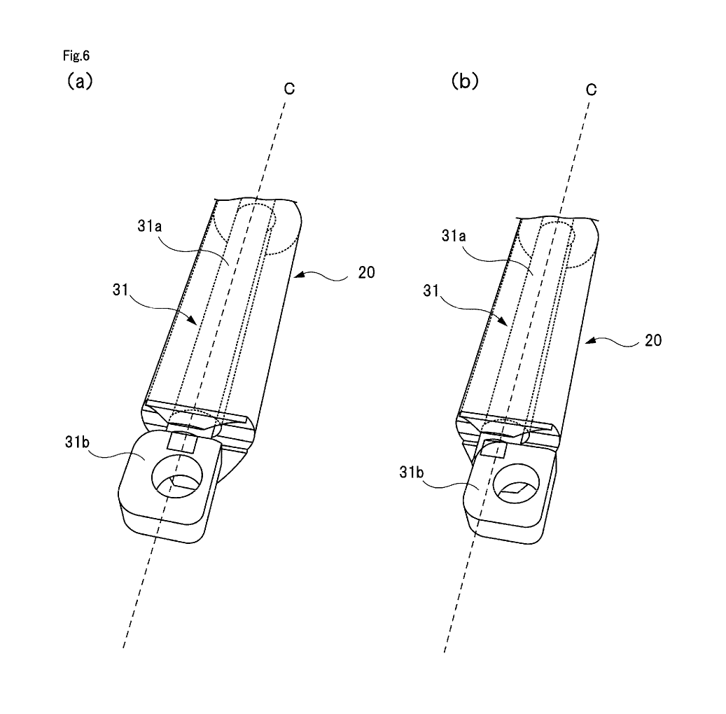

FIGS. 6a and 6b are figures for explaining an improvement example of a peritoneal peeling surgical instrument 100 according to the present invention. As illustrated in FIGS. 6a and 6b, a ring part 31b having a round hole through which the connection pin 32 is to be put is disposed at the tip of the slide shaft 31. In the example illustrated in FIG. 6a, the round hole of the ring part 31b is located on the central axis C of the slide shaft 31. On the other hand, in the example illustrated in FIG. 6b, the round hole of the ring part 31b is deviated from the central axis C of the slide shaft 31. In the peritoneal peeling surgical instrument 100 of the present invention, it is more preferable to employ the embodiment in FIG. 6b than to employ the embodiment in FIG. 6a. That is, the round hole of the ring part 31b corresponds to the rotation fulcrum of the hook part 40. Thus, when the round hole (rotation fulcrum) of the ring part 31b is deviated from the central shaft C of the slide shaft 31 as illustrated in FIG. 6b, the hook part 40 is bent with respect to the shaft part 20 so that the strength of the state can be improved. This makes it possible to maintain the bending state of the hook part 40 stably even when a larger load is applied to the hook part 40 in a bent state. Specifically, the round hole (rotation fulcrum) of the ring part 31b is preferably deviated from the central shaft C of the slide shaft 31 in a direction opposite to the direction in which the hook part 40 is bent. Here, it is clear that the embodiment in FIG. 6a also can be employed in the peritoneal peeling surgical instrument 100 of the present invention.

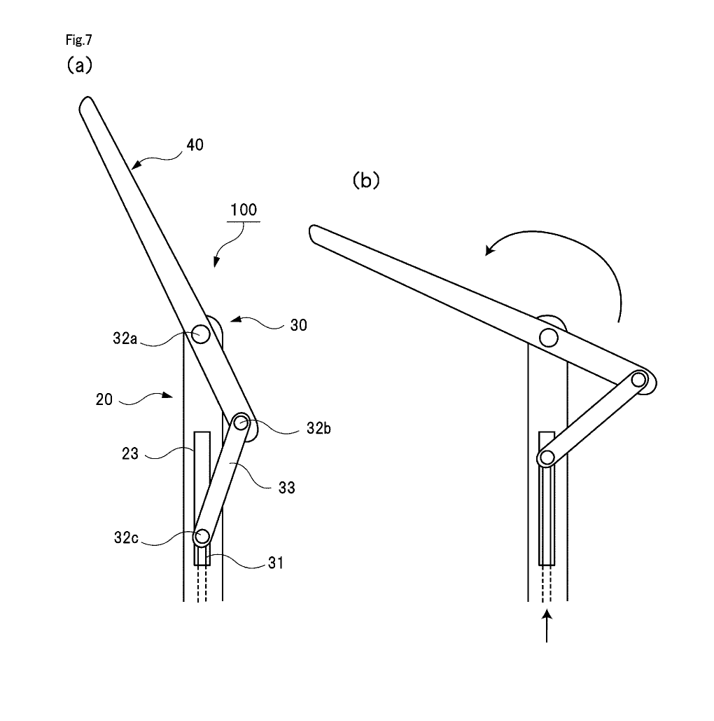

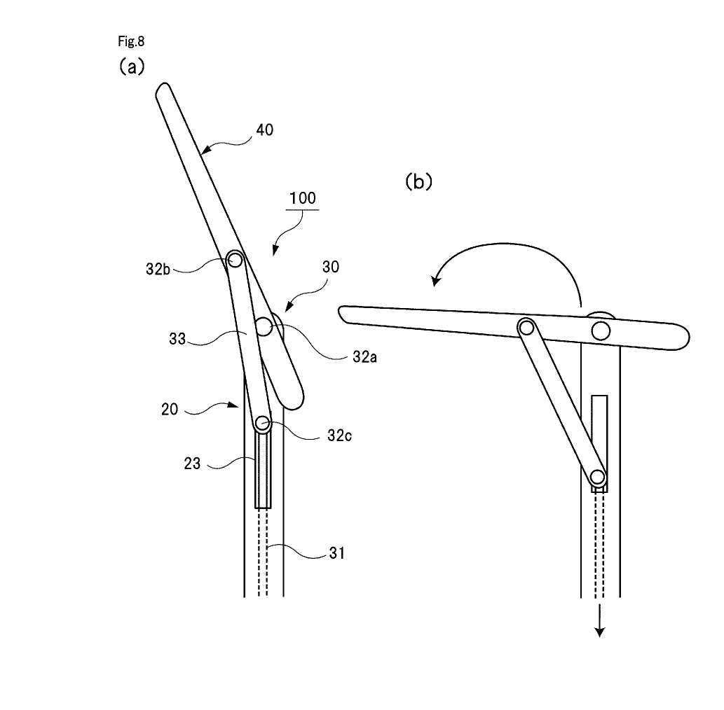

In the present invention, the structure of the hinge part 30 is not limited to the structure described above. For example, other embodiments of the hinge part 30 are illustrated in FIGS. 7a to 8b.

In the embodiment illustrated in FIGS. 7a and 7b, the hook part 40 is rotatably connected with the tip of the shaft part 20 by a first connection pin 32a which functions as a rotation fulcrum. Moreover, a second connection pin 32b is attached to a position of the hook part 40 closer to the base end than the first connection pin 32a. The hook part 40 is rotatably connected with the crank member 33 by the second connection pin 32b. One end of the crank member 33 is connected with the base end side of the hook part 40 by the second connection pin 32b, and the other end of the crank member 33 is connected with the tip of the slide shaft 31, which is advanced and retracted in the shaft part 20, by a third connection pin 32c. Moreover, a slide hole 23 to be used for regulating the movement direction of the third connection pin 32c is formed at the shaft part 20. This causes the third connection pin 32c to advance and retract straight along the slide hole 23 of the shaft part 20.

When the slide shaft 31 is advanced toward the tip side in such a structure, the hook part 40 is bent with respect to the shaft part 20 with the first connection pin 32a as a rotation fulcrum as illustrated in FIG. 7b. Such a simple structure also can realize the hinge part 30 of the peritoneal peeling surgical instrument 100.

In the embodiment illustrated in FIGS. 8a and 8b, the hook part 40 is rotatably connected with the tip of the shaft part 20 by a first connection pin 32a which functions as a rotation fulcrum. Moreover, a second connection pin 32b is attached to a position of the hook part 40 closer to the tip than the first connection pin 32a. The hook part 40 is rotatably connected with the crank member 33 by the second connection pin 32b. One end of the crank member 33 is connected with the base end side of the hook part 40 by the second connection pin 32b, and the other end of the crank member 33 is connected with the tip of the slide shaft 31, which is advanced and retracted in the shaft part 20, by a third connection pin 32c. Moreover, a slide hole 23 to be used for regulating the movement direction of the third connection pin 32c is formed at the shaft part 20. This causes the third connection pin 32c to advance and retract straight along the slide hole 23 of the shaft part 20.

When the slide shaft 31 is retracted toward the side closer to the user in such a structure, the hook part 40 is bent with respect to the shaft part 20 with the first connection pin 32a as a rotation fulcrum as illustrated in FIG. 8b. Such a simple structure also can realize the hinge part 30 of the peritoneal peeling surgical instrument 100.

Regarding the present invention, material to be used for the peritoneal peeling surgical instrument 100 is not limited especially. In order to provide a peritoneal peeling surgical instrument 100 which can be cleaned and reused, the instrument 100 may be made of metal such as stainless steel. In order to provide a disposal peritoneal peeling surgical instrument 100, the instrument 100 may be made of resin such as plastic. For example, the peritoneal peeling surgical instrument 100 according to the embodiment illustrated in FIGS. 1a to 1c and the like is preferably made of metal so as to be cleaned and reused. On the other hand, the peritoneal peeling surgical instrument 100 according to the embodiments illustrated in FIGS. 7a to 8b and the like is preferably made of resin to be disposal.

The foregoing description of this specification has explained embodiments of the present invention with reference to the drawings in order to express the content of the present invention. Here, the present invention is not limited to the above embodiments but includes modified embodiments or improved embodiments which are clear to those skilled in the art on the basis of the matters described in this specification.

Reference Signs List

| | | | |
|---|---|---|---|
| 10 | Operation part | 11, 12 | Handle member |
| 13 | Fulcrum member | 14, 15 | Crank member |
| 20 | Shaft part | 21 | Level surface |
| 22 | Inclined surface | 23 | Slide hole |
| 30 | Hinge part | 31 | Slide shaft |
| 31a | Elongate cylindrical shaft part | 31b | Ring part |
| 32 | Connection pin | 32a | First connection pin |
| 32b | Second connection pin | 32c | Third connection pin |
| 33 | Crank member | 40 | Hook part |
| 41 | Convex curved surface | 42 | Flat surface |
| 43 | Streamline tip part | | |
| 100 | peritoneal peeling surgical | | |
| | instrument | | |

1. A peritoneal peeling surgical instrument, which is put into a space between muscle and a peritoneum via a tunnel formed at the peritoneum and is used to peel the peritoneum from the muscle, comprising:
an operation part (10) which is grasped and operated by a user;
a shaft part (20) connected with the operation part (10);
a hinge part (30) disposed at a tip of the shaft part (20); and
a hook part (40) for peeling connected with the shaft part (20) via the hinge part (30),
wherein the hook part (40) is bent at the hinge part (30) with respect to the shaft part (20) in response to operation to the operation part (10) and a bending state of the hook part (40) is fixed in one or a plurality of steps, and
wherein the hook part (40) has:
a convex curved surface (41) located at a bending direction side which can be in contact with the peritoneum;
a flat surface (42) located at a bending direction opposite side which can be in contact with the muscle; and
a streamline tip part (43) corresponding to a tip of the hook part (40).

2. The peritoneal peeling surgical instrument according to claim 1, wherein the bending state of the hook part (40) is fixed at least at a first step bent by 120±10° with respect to the shaft part (20), and a second step bent by 90±10° with respect to the shaft part (20).

3. The peritoneal peeling surgical instrument according to claim 1, wherein
the hinge part (30) has a slide shaft (31), wherein the a slide shaft (31) is a baculiform member having one end part connected with the operation part (10) and the other end part rotatably connected with the hook part (40) and can be advanced and retracted along the shaft part (20) in response to operation to the operation part (10),
a connection state between the shaft part (20) and the hook part (40) is released and the hook part (40) becomes rotatable when the slide shaft (31) is advanced, and
the shaft part (20) and the hook part (40) are connected with each other and the hook part (40) is fixed to the shaft part (20) when the slide shaft (31) is retracted.

4. The peritoneal peeling surgical instrument according to claim 3, wherein the hook part (40) has rigidity to withstand a load even in a case where a load of 500 g is applied to a tip part of the hook part (40) in the process of peeling the peritoneum in a state where the hook part (40) is fixed to the shaft part (20).

5. The peritoneal peeling surgical instrument according to claim 3, wherein a connection pin (32) configured to rotatably connect the slide shaft (31) and the hook part (40) with each other is formed at a position deviated from a central axis (C) of the slide shaft (31).
